(19) United States
(12) Patent Application Publication
(10) Pub. No.: US 2005/0171407 A1
(43) Pub. Date: Aug. 4, 2005

(54) Illumination device and method for medical procedures

(76) Inventors: Michael Rosenkranz, Seevetal (DE); Wolfgang Daum, Groton, MA (US)

Correspondence Address: Wolfgang Daum, 20 Whiley Road, Groton, MA, 01450, US

(21) Appl. No.: 10/988,296

(22) Filed: Nov. 13, 2004

Related U.S. Application Data
(60) Provisional application No. 60/524,609, filed on Nov. 24, 2003.

(51) Int. Cl. A61B 1/04

(52) U.S. Cl. 600/249

(57) ABSTRACT

A method and device to illuminate medical sites using light emitting diodes (LED) and bendable mechanical arms (goosenecks) to carry the arrays of LED's. The device is battery powered.

### FIELD OF THE INVENTION

The invention relates to interventional medicine and surgery. Specifically, the invention provides a method and an illumination device to illuminate areas of interest during surgery, minimally invasive surgery or medical interventions. The device is battery powered and comprises at least one flexible goose neck arm and at least one laser diode light source.

### BACKGROUND OF THE INVENTION

For diagnostic examination, interventional and surgical procedures physicians and nurses need light. Various light sources have been introduced, which can be permanently fixed to walls, ceilings or floors or are movable with and without wheels. Those lamps generally use light bulbs or arc discharge light sources.

The disadvantage of a lot of light systems is, that they shine from far away onto the medical site of interest. When the physician is working under those light conditions shadow of the physician might darken the site. Because conventional lamps are rather large in size and bulky, the light has to be illuminated from far away or guided by glass fiber. Jesurun has disclosed in U.S. Pat. No. 6,601,985 a medical light system, which guides light from a metal halide lamp to the surgical sight. However, the glass fiber construction is rather costly.

The aim of this invention is to disclose an inexpensive to produce medical light system, which illuminates the medical site shadow less and can be used for all medical modalities.

Krenzel RE36,883 has disclosed a holder for a flash light. This invention provides an elongated flexible gooseneck design that is capable of being formed into a plurality of differing shapes so that it can be supported at a variety of locations and has retaining means for retaining it in a desired shape so that a holder connected to one end thereof can hold a flashlight at any desired position relative thereto. In one embodiment of the invention Krenzel uses a flexible gooseneck of company Lockwood. Also Cedarberg III has disclosed a design D392,758, which is mend to be a flash light holder. In combination with a light emitting diode (LED) flashlight of for instance of Parker U.S. Pat. No. 6,536,912 a new flexible LED based gooseneck lamp could be designed. However, such a lamp design would be desolate and does not comprise a stable device stand and robust device design to sustain the often harsh and rough clinical routines. Such a device would be vulnerable to being damaged upon flexing of the gooseneck.

Altman U.S. Pat. No. 6,004,004 disclosed a dual flashlight assembly, which could be combined with LED light sources to give better illumination of the areas of interest during medical procedure. However, because a design such as this would have to be temporarily mounted on any type of stand it would also be desolate and not procedure dedicated. There is a need for a multiple medical light assembly, which is either mounted directly to the room (ceiling, wall, floor) or has it's own stable stand.

At present the problem with existing surgical flood lights is, that they are mounted behind the physician or behind the head of the physician and the shadow of the physician can hinder the work.

### BRIEF SUMMARY OF THE INVENTION

The present invention provides an illumination device to closely generate light out of small light emitting diodes (LED's) next to the patients area of interest. The light source (LED's) are mounted on totally flexible and hand movable arms, also known as goose necks. In opposite to surgical flood lights, the invented illumination device brings the light source with the help of at least one gooseneck to the front side of the physician close to the to be illuminated medical site. Hence, no shadow will hinder the work of the physician. In order to avoid trapping over cables, the illumination device is battery powered.

### BRIEF DESCRIPTION OF THE DRAWINGS

FIG. 1 illustrates various LED arrangements. ° 0101 FIG. 2 illustrates an example of a gooseneck.

FIG. 3 illustrates various illumination device arrangements.

FIG. 4 illustrates a battery powered stand alone illumination device.

FIG. 5 illustrates the stand of the illumination device of FIG. 4.

FIG. 6 illustrates the battery pack detail of the illumination device of FIG. 4.

FIG. 7 illustrates an electrical circuit board of the illumination device of FIG. 4.

FIG. 8 illustrates the current regulator circuit for the LED's of illumination device of FIG. 4.

FIG. 9 illustrates a power load circuit for batteries of illumination device of FIG. 4.

FIG. 10 illustrates one position of the illumination device relative to the patient.

FIG. 11 illustrates an illumination device, which can be attached to a some other device.

### NUMBERS

1 Light Emitting Diode (LED)
2 LED
3 LED
4 diode array plate with one LED
5 diode array plate with symmetrical arranged LEDs
6 diode array plate with arbitrary arranged LEDs
7 gooseneck element or gooseneck segment
8 distal gooseneck element onto or in which the diode array plate is mounted.
9 proximal gooseneck element or goose neck socket
10 gooseneck
11 ball end of the gooseneck element
12 socket end of the gooseneck element
13 room
14 ceiling
15 wall
16 floor
17 gooseneck
18 ceiling mounting socket for a single gooseneck
19 LED array
20 ceiling socket for multiple goosenecks
21 first gooseneck
22 second gooseneck
23 third gooseneck
24 first LED array
25 second LED array
26 third LED array
27 wall mounting socket for gooseneck
28 main gooseneck
29 T type or Y type branch fitting
30 gooseneck branch
31 LED array of gooseneck branch
32 LED array of main gooseneck
33 floor mounting socket
34 post
35 gooseneck
36 LED array
37 movable battery powered medical gooseneck lamp
38 wheels of 37
39 post 37
40 gooseneck
41 LED array
42 battery pack
100 illumination device
101 stand
102 five-foot-stand (tripod) with wheels
103 wheels
104 base of stand 101 with battery box or also called base tube
105 tube bend at the top
106 y connector to gooseneck elements 107
107 gooseneck
108 LED array
109 connector between base of stand 104 and tube of stand 105
110 electrical switch
111 closure of base 104
112 floor
113 upper height
114 bending angle of tube 105
115 bended section of tube 105
116 distal end of tube 105
117 battery pack
118 Screw
119 circuit board for power source
120 electrical jack for charging transformer
121 electrical plug
122 chargeable batteries
123 current regulator for LED array 129
124 current regulator for LED array 128
125 power load circuit or recharge circuit
126 transformer or switching power supply
127 plug for AC
128 LED array
129 LED array
130 switch
131 LED array or single LED
132 voltage measurement point
133 transistor
134 Zener diode
135 resistor
136 resistor
137 battery pack
138 measurement point
139 input measurement point
140 to be recharged batteries
141 transistor
142 output measurement point
143 diode
144 diode
145 resistor
146 Zener diode
147 resistor
148 input measurement point
149 diode
150 resistor
152 output measurement point
153 illumination device
154 gooseneck
155 light source
156 electric box
157 adapter
158 physician
159 patient
160 illumination device

### DETAILED DESCRIPTION

1. General Description

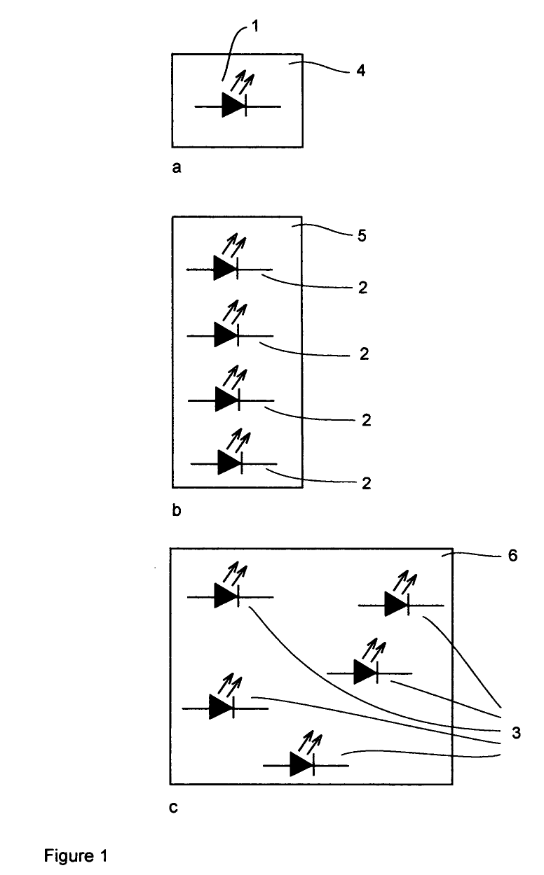

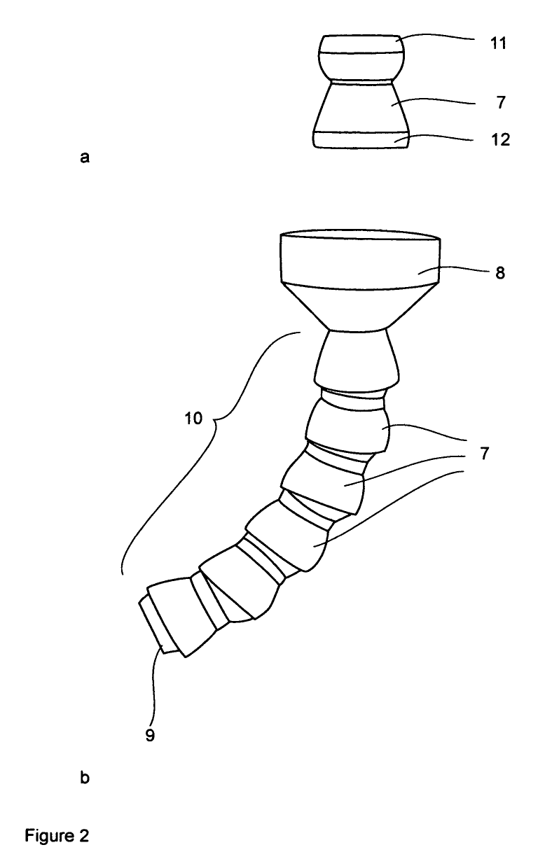

In FIG. 1a is shown a light emitting diode (LED) 1 in form of the electric symbol. The diode is mounted on the diode array 4. Other configurations of such LED arrays are shown in FIG. 1b, where the diodes 2 are mounted in a symmetrical way on the array plate 5, and FIG. 1c, where the diodes 3 are mounted in an arbitrary arrangement on the array plate 6. Sometimes such LED arrays are also called LED cluster, LED assemblies or LED panels.

Goosenecks are flexible arms which can be bend in various directions and shapes. Due to the relative tight friction between the gooseneck elements the shape or bending of the gooseneck will not change. FIG. 2a shows a gooseneck element 7 with ball 11 and socket 12.

A gooseneck as described and used here is an assembly of a plurality of similar elements, whereas each one element fits relatively tight with it's front part into the back part of a second element, to give an overall elongated arm design. Although the elements fit relatively tight into each other, they can still be moved or bend against each other by hand operation to give a rigid but flexible structure to be shaped in different direction. A gooseneck comprises more than one and less than 100, more typically between 10 and 30 elements.

In FIG. 2b is shown an example of a gooseneck 10. The elements 7 (or segments 7) of the gooseneck 10 are made out of plastic or metal. Plastic as polypropylene (PP), polyethylene (PE), polyvinylechloride (PVC), Teflon, PEEK or others can be used. Metals like stainless steel, titanium, aluminium, brass or any metal-alloys can be used. Good plastic goosenecks out of PVC are available from company Lockwood Products, Inc., 5615 SW Willow Lane, Lake Oswego, Oreg. 97035, USA. See also the corresponding patents of Mark B. Lockwood U.S. Pat. No. 5,449,206 and U.S. Pat. No. 6,042,155, or see the patent of Gregory G. Johnson U.S. Pat. No. 5,921,204. The advantage of plastic elements 7 are that they are washable and acid or base resistant. One could also use the above mentioned design of Cedarberg III D392,758 as one part of the illumination device here.

On or in the distal gooseneck element 8 is mounted the LED array. The gooseneck comprises a socket 9 on the proximal end, with which it is mounted somewhere.

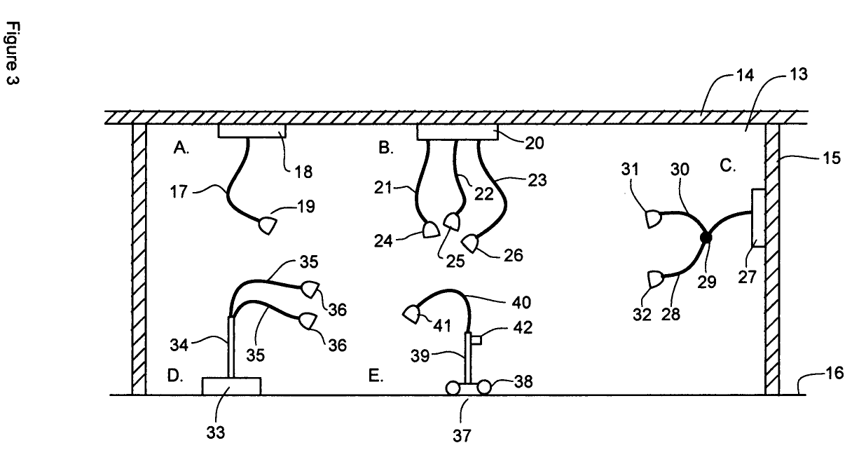

FIG. 3 shows various types of illumination devices as they might be used for medical applications. A, B, C and D are illumination devices which are fixed in the room. A is a ceiling 14 mounted device with an LED array 19, gooseneck 17 and ceiling mounting socket 18. This device A will be like all others moved by hand to bring it in the right position.

Device B is an illumination device which is also mounted to the ceiling 14 of the room 13 with a ceiling mounting socket 20. In this device B three different goosenecks 21, 22 and 23 are holding three different LED arrays 24, 25 and 26. The difference in the goosenecks are length, diameter, material or colour. The difference in LED arrays are number of LEDs per array, diode light output of the LEDs, emitting angle, colour of the LED light and arrangement of the LEDs on the array plate.

Illumination device C is a wall 15 mounted device with wall mounting socket 27, main gooseneck 28 with main LED array 32 and an additional gooseneck branch 30 with LED array 31. The branch 30 branches off the main gooseneck with a T- or Y-type branch fitting.

Illumination device D is a floor 16 mounted device, at which two identical goosenecks 35 with identical LED arrays 36 are mounted on a post 34.

Example 37 shows a movable type of illumination device on wheels 38. The gooseneck 40 with LED array 41 is mounted on a post 39. The whole device is powered from a battery in a battery box 42.

It would be obvious for someone skilled in the art to find any other combination of goosenecks and LED arrays. Those devices could also be mounted on medical equipment such as radiological X-ray, ultrasound or MRI machines, surgical tables, chairs etc. Company Lookwood provides various parts to find different arrangements. The various parts in the Lookwood Products Inc. catalogue of September 200 (form no. 99083) are herewith incorporated by reference.

2. Stand Alone Battery Powered Illumination Device

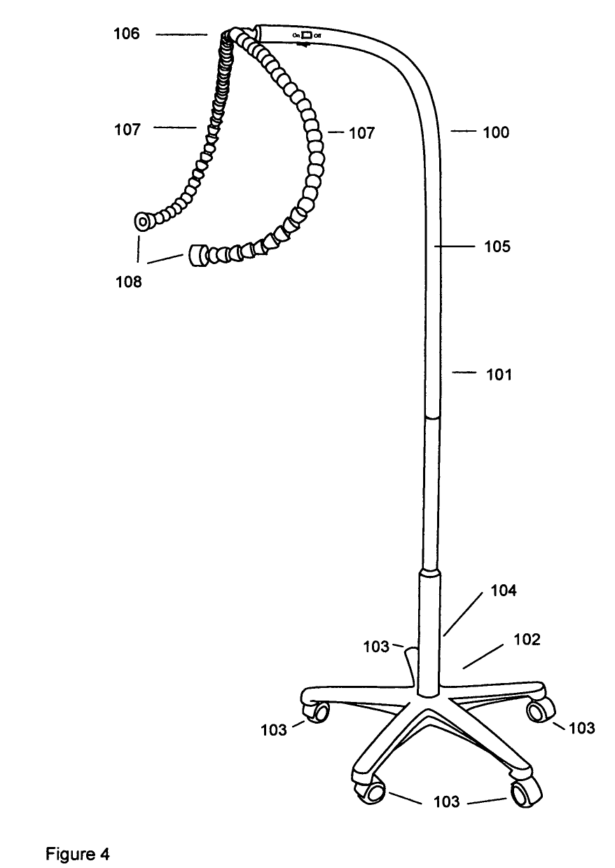

FIG. 4 shows an stand alone illumination device 100 comprising a stand 101, which is mounted on a base 104 having a five-leg-base (tripod) 102 with wheels 103. In the base 104 are the batteries and some electrical circuits. The stand 101 is made in it's upper part from a tube 105 which bends at its top. A y connector 106 at the top end of the tube 105 connects to two identical gooseneck arms 107, those carrying the LED arrays 108 at their end.

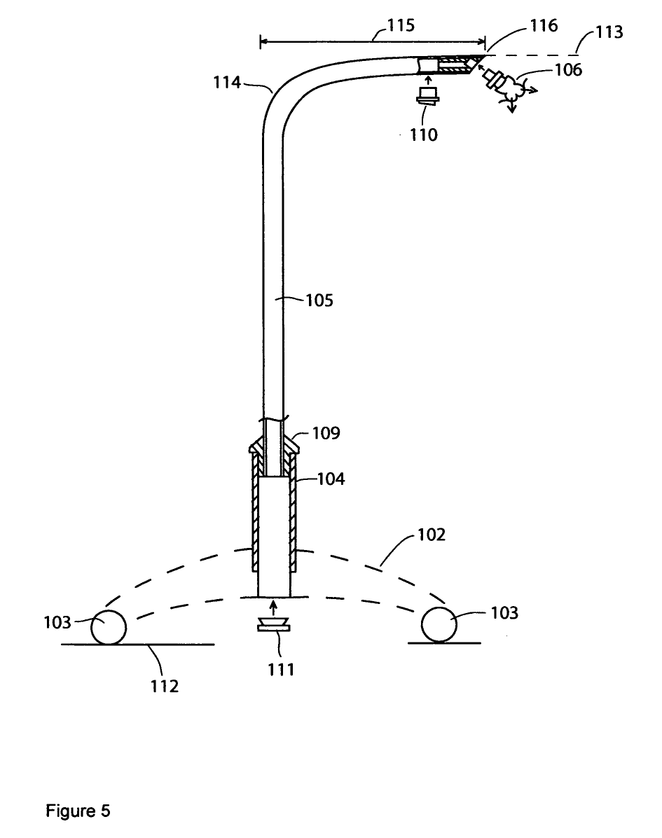

The height for the illumination device from the floor 112 to the upper height 113 varies from 500 to 2,500 millimetres, typically the height is 1,700 millimetres. In one embodiment of the invention this height is adjustable. The bending angle 114 of the tube 105 varies from 0° to 180° and is typically 90°. The thickness of the tube 105 varies between 10 and 50 millimetres and is typically about 30 millimetres. The wall thickness of tube 105 varies between 0.5 and 5.0 millimetres and is typically 2 millimetres. The base 104 is a tube, with diameter varying between 20 and 60 millimetres, typically 50 millimetres. The wall thickness of base tube 104 varies between 0.5 and 5.0 millimetres and is typically 2 millimetres. The arm length of the bended section of tube 105 varies in length between 100 and 1,000 millimetres and is typically 400 millimetres long. The distal end 116 can be cut straight or cut in an angle as shown in FIG. 5. The tubes 104 and 105 as well as the connector 109 are made from aluminium. These could also be made from stainless steel, steel, brace, wood, or any type of plastic. These parts can be specially surface treated by oxidation or painting.

In FIG. 5 a tripod is used. It would be obvious for someone skilled in the art to use any other number of legs for the foot of the device. There could be 4, 5 or more wheel-legs. The foot does not even have to have wheels. The wheels comprise breaks to prevent the device from freely rolling. There might be more or less than 2 goose arms 107. The goose arms 107 might be different in length and diameter. The material for the tripod 102 or wheels 103 is aluminium, steel, stainless steel, brace, plastic or wood.

In this device of FIG. 5 a switch 110 is mounted at the distal part 116 of the tube 105, switching the device on and off. There could also be a dimmer, not shown here.

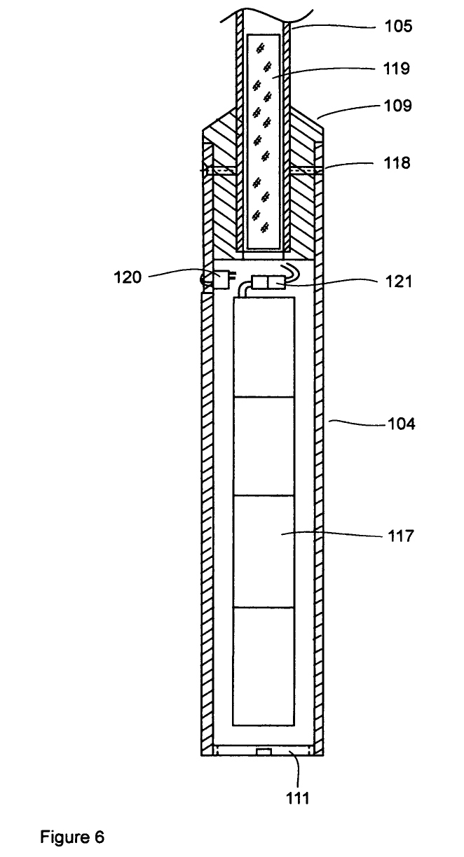

In FIG. 6 it is shows that is the base tube 104 is the located the battery pack 117. The battery pack 117 consists of four D sized batteries, connected in series and connected to the power circuit board 119 by an electrical plug 121. It would be obvious for someone skilled in the art to use different size type batteries here. The battery pack 117 can be removed by opening the shutter 111. In this case the shutter 111 is a round metallic plate with a thread to be screwed into the base tube 104. Tube 105 and 104 are connected with the connector 109 by screws 118. Electrical jack 120 connects the device to a charging transformer if needed. In usual operation the device is left alone and powers the LED's from the batteries 117.

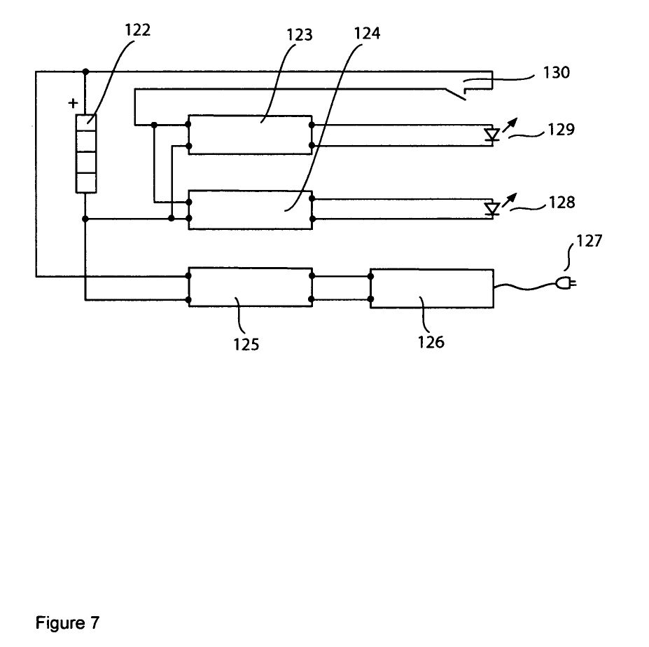

FIG. 7 is the basic electrical block circuit diagram of the circuitry of the device. Current regulators 123 and 124 supply the LED arrays 128 and 129 with constant DC current from batteries 122. Batteries 122 in this case are rechargeable by power load circuit 125, which gets its power from switching power transformer 125 of external power supply 127. Typically the input voltage for the transformer 126 is from 90 to 230 volts and 50 to 60 hertz (Hz) frequency. The typical output voltage of the transformer is 9 volts. The LED 128 and 129 are switched on or off with switch 130 (110 in FIG. 5). Batteries 122 are typically NiMH (Nickel-metal-hydrate) rechargeable batteries of 4.8 to 5.6 volts and 7 ampere-hours. It would be obvious for someone skilled in the art to use different types of batteries. NiMH batteries show no memory effect.

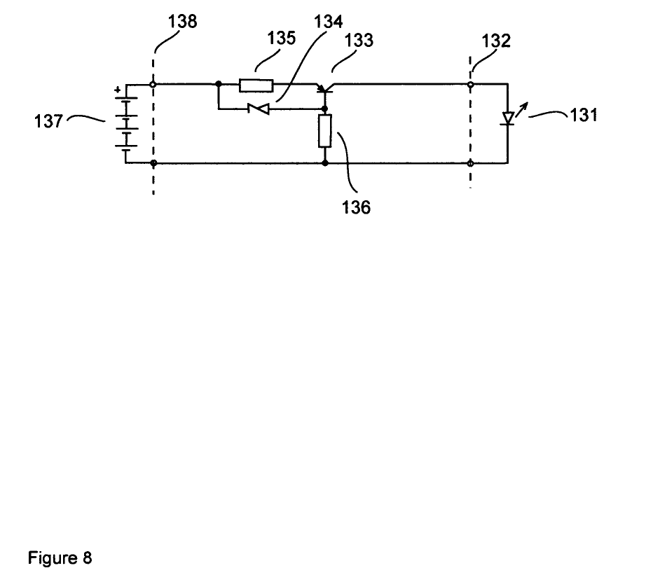

Current regulators 123 and 124 are shown in detail in FIG. 8. Light emitting diodes 131 would be typically a white NSPW500BS of company NICHIA, see www.eska-technik.com with luminous intensity of 11 candela and emitting angle of ±10°, or a LUXEON Star/0 LXHL-NW98, see for instance www.globalspec.com. The NiMH battery pack 137 gives typically 4.8 to 5.6 volts at 7 ampere-hours at measurement point 138. Typically transistor 133 is a BD132 type, Zener diode 134 is a 1.5 volt type, resistor 135 is a 3.6 ohms at 0.5 watt type, and resistor 136 is a 390 ohms at 0.1 watts type. At measurement point 132 a constant current of 230 milliamps with 3.5 volts can be measured. It would be easy for someone skilled in the art to use different types of LED'S and design appropriate current regulating circuits for these.

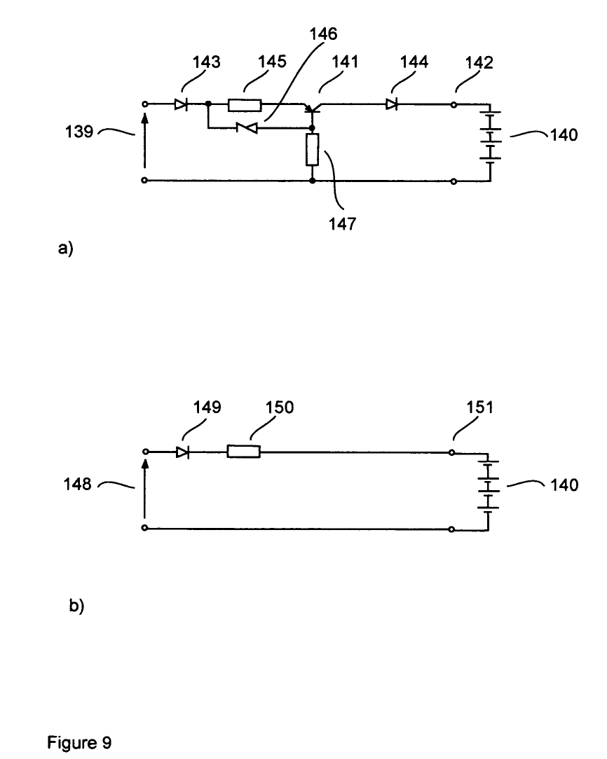

FIG. 9 illustrates two possible power load circuits to recharge the batteries. In FIG. 9a is shown a current regulator circuit charging batteries 140 of 4.8 to 5.6 volts. At input measurement point 139 9 volts and at output measurement point 142 a constant current of 350 milliamps are measured. Typically transistor 141 is a BD 132, resistor 145 is a 2.4 ohms at 0.5 watt and resistor 147 is a 390 ohm at 0.1 watt, diode 143 and 144 are 1N4001 and Zener diode 146 is a 1.5 volts type. Circuit as shown in FIG. 9b is easier and only recharges the batteries in the amount as they unload over time and will transform the 9 volts at input 148 to a constant current of 350 milliamps at output 151 for batteries 140. Typically diode 149 is of 1N4001 and resistor 150 of 9.1 ohms at 2 watts type. The circuit in FIG. 9b does not use an “intelligent” design because for this circuit the defined current is not critical.

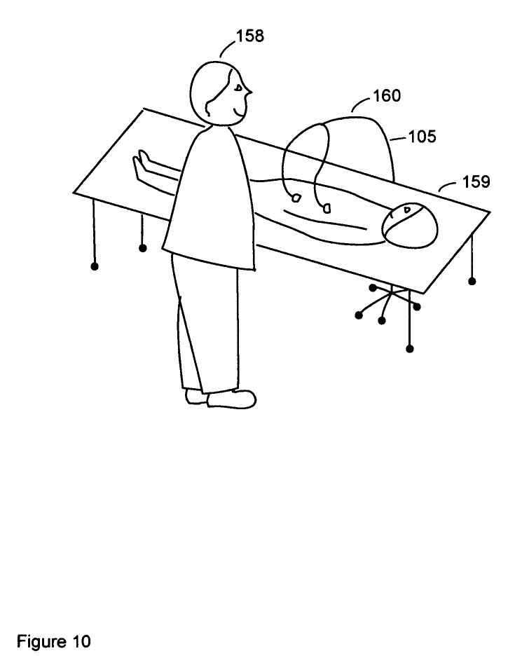

FIG. 10 shows how the patient 159 would be positioned between the physician 158 or nurse and the illumination device 160. The tube 105 would reach over the patient and the physician could bend the goosenecks by hand to such a shape that the LED arrays would illume the site of interest optimal.

It would be obvious for someone skilled in the art to build such a stand alone battery powered illumination device with any other number of goose arms, such as one, three, four, five, etc.

3. Adaptable Battery Powered Illumination Device

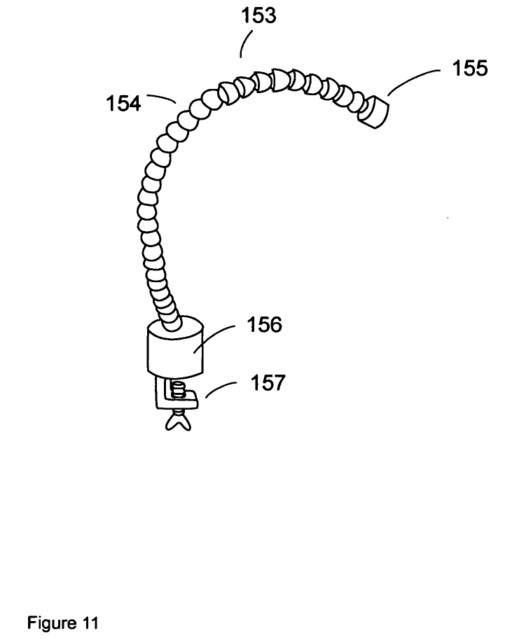

FIG. 11 shows an illumination device 153 comprising a goose neck 154, a LED light source 155 of the kind described elsewhere in here, an electric box 156, in which the electric circuitry and battery pack in located, and an adapter 157 to adapt the illumination device 153 to any other device. With this adapter 157 the illumination device 153 can be adapted to a patient bed, a patient table, a patient chair, an operational table, an interventional table, a dentist patient chair, a gynaecological patient chair, a neurological stereotactic frame or the like.

1. A method to illuminate a site to perform a medical procedure comprising at least one light emitting diode (LED) mounted on the distal ends of at least one gooseneck and bending such gooseneck by hand so that the LED illuminates the site.

2. A method according to claim 1, whereas the LED is battery powered.

3. A method according to claim 1, whereas the gooseneck is permanently mounted to a part of the room.

4. A method according to claim 1, whereas the gooseneck is mounted on medical equipment.

5. A method according to claim 1, whereas the goosenecks is mounted on a movable stand.

6. A method according to claim 5, whereas the stand comprises wheels.

7. A method according to claim 1, wherein the gooseneck reaches over the patient to the physician to be bend in the desired position.

8. A device to illuminate a medical site comprising at least one light emitting diode (LED) mounted on the distal ends of a gooseneck.

9. A device according to claim 8, whereas the number of goosenecks per device is in between two and eight.

10. A device according to claim 8, whereas the material of the goosenecks is at least one element of the group consisting of plastic, metal, metal alloy, polypropylene (PP), polyethylene (PE), polyvinyl chloride (PVC), Teflon, PEEK, stainless steel, titanium, aluminium, brass or any combination from these.

11. A device according to claim 8, whereas the number of elements of one gooseneck is between 5 and 40.

12. A device according to claim 8, whereas the LED's are powered by rechargeable batteries.
